(19) United States
(12) Patent Application Publication
(10) Pub. No.: US 2011/0138882 A1
(43) Pub. Date: Jun. 16, 2011

(54) SEMICONDUCTOR GAS SENSOR HAVING LOW POWER CONSUMPTION

(75) Inventors: Seung Eon Moon, Daejeon (KR); Jong Hyurk Park, Daegu (KR); Jae Woo Lee, Daejeon (KR); Hong Yeol Lee, Chungcheongbuk-do (KR); Kang Ho Park, Daejeon (KR); Jong Dae Kim, Daejeon (KR)

(73) Assignee: ELECTRONICS AND TELECOMMUNICATIONS RESEARCH INSTITUTE, Daejeon (KR)

(21) Appl. No.: 12/965,433

(22) Filed: Dec. 10, 2010

(30) Foreign Application Priority Data
Dec. 11, 2009 (KR) 10-2009-0123175
Nov. 22, 2010 (KR) 10-2010-0116063

(51) Int. Cl. G01N 33/00; H01L 21/368; B82Y 15/00

(52) U.S. Cl. 73/31.06; 438/49; 257/E21.464; 977/957

(57) ABSTRACT

Provided are a structure and operating method of a semiconductor gas sensor having low power consumption. The semiconductor gas sensor is adapted to adsorb gas to a low-dimensional semiconductor nanomaterial at room temperature, output a change in resistance of the low-dimensional semiconductor nanomaterial, apply power to a heater, desorb the gas adsorbed to the low-dimensional semiconductor nanomaterial, and return the resistance of the low-dimensional semiconductor nanomaterial back to initial resistance. The semiconductor gas sensor senses the gas at room temperature using the low-dimensional semiconductor nanomaterial having a high-sensitivity characteristic at room temperature, and drives the heater only when the adsorbed gas is desorbed. Thereby, it is possible to improve a gas sensing characteristic, reduce power consumption, and provide a rapid response speed.

### CROSS-REFERENCE TO RELATED APPLICATION

This application claims priority to and the benefit of Korean Patent Application Nos. 10-2009-0123175 filed Dec. 11, 2009, and 10-2010-0116063 filed Nov. 22, 2010, the disclosure of which is incorporated herein by reference in its entirety.

### BACKGROUND

1. Field of the Invention

The present invention relates to a gas sensor, and more particularly, to a semiconductor gas sensor using a micro electromechanical system (MEMS).

2. Discussion of Related Art

Gas sensors have been studied for a long time. Many types of gas sensors are currently commercialized.

Among them, a gas sensor using a semiconductor is based on a principle that, when gas component reacts with adsorption gas such as oxygen, which has been previously adsorbed or is adsorbed on a surface of the semiconductor, electron exchange occurs between adsorbed molecules and the semiconductor surface, and thus the characteristics of the semiconductor such as conductivity and surface potential are subject to variation. This variation is detected by the gas sensor.

Such semiconductor gas sensors have a simple structure, an easy process, a small size, and low power consumption, compared to optical gas sensors or electrochemical gas sensors which measure conductivity caused by a spectrum or ion mobility of air to be measured.

The semiconductor gas sensors use a heater formed on the rear surface of a substrate to increase a surrounding temperature to smoothly sense gas. Thus, the semiconductor gas sensors have about 30 to 100 times as high power consumption as other commercialized sensors such as temperature or humidity sensors, which have power consumption in the order of mW. As such, it is difficult to apply the semiconductor gas sensors to various services using a ubiquitous sensor network or portable terminals. There is a need for the semiconductor gas sensor having low power consumption.

### SUMMARY OF THE INVENTION

The present invention is directed to a semiconductor gas sensor in which power consumption is remarkably reduced.

The present invention is also directed to a semiconductor gas sensor having a rapid response characteristic.

Other objectives of the present invention may be understood from the following description and exemplary embodiments of the present invention.

One aspect of the present invention provides a semiconductor gas sensor having low power consumption, which adsorbs gas to a low-dimensional semiconductor nanomaterial at room temperature, outputs a change in resistance of the low-dimensional semiconductor nanomaterial, applies power to a heater, desorbs the gas adsorbed to the low-dimensional semiconductor nanomaterial, and returns the resistance of the low-dimensional semiconductor nanomaterial back to initial resistance.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a cross-sectional view of a semiconductor gas sensor according to an exemplary embodiment of the present invention;

FIG. 2 is a graph showing measuring sensitivity of a semiconductor gas sensor according to an exemplary embodiment of the present invention; and

FIG. 3 is a graph showing measuring sensitivity of a semiconductor gas sensor according to another exemplary embodiment of the present invention.

### DETAILED DESCRIPTION OF PREFERRED EMBODIMENTS

The present invention will now be described more fully hereinafter with reference to the accompanying drawings, in which exemplary embodiments of the invention are shown. Throughout the drawings, it should be noted that the same reference numerals or symbols are used to designate like or equivalent elements having the same function. The detailed descriptions of known function and construction unnecessarily obscuring the subject matter of the present invention will be omitted. Technical terms, as will be mentioned hereinafter, are terms defined in consideration of their function in the present invention, which may be varied according to the intention or practices of user or operator, so that the terms should be defined based on the contents of this specification.

As described above, semiconductor gas sensors in the related art are designed to sense gas and desorb adsorbed gas at high temperature, and thus have higher power consumption than other sensors (e.g., temperature sensors or humidity sensors). As such, the semiconductor gas sensors have difficulty in application to various services using a ubiquitous sensor network, and are subject to reduction in durability due to high-temperature operation.

Thus, to overcome these problems, the present invention provides a semiconductor gas sensor, in which low-dimensional nanomaterials, such as nanopowder, nanowires, nanorods, carbon nanotubes (CNTs), and graphene, which have a large surface area per volume and a good gas sensing characteristic at room temperature, are used as gas sensing materials, and in which a heater is driven only when gas is desorbed.

In detail, the semiconductor gas sensor is adapted to adsorb gas to a low-dimensional semiconductor nanomaterial at room temperature, output a change in resistance of the low-dimensional semiconductor nanomaterial, apply power to a heater, desorb the gas adsorbed to the low-dimensional semiconductor nanomaterial, and return the resistance of the low-dimensional semiconductor nanomaterial back to initial resistance.

The semiconductor gas sensor of the present invention exhibits a low-power characteristic and a rapid response characteristic compared to the existing sensors.

Hereinafter, exemplary embodiments of the present invention will be described in detail with reference to the accompanying drawings.

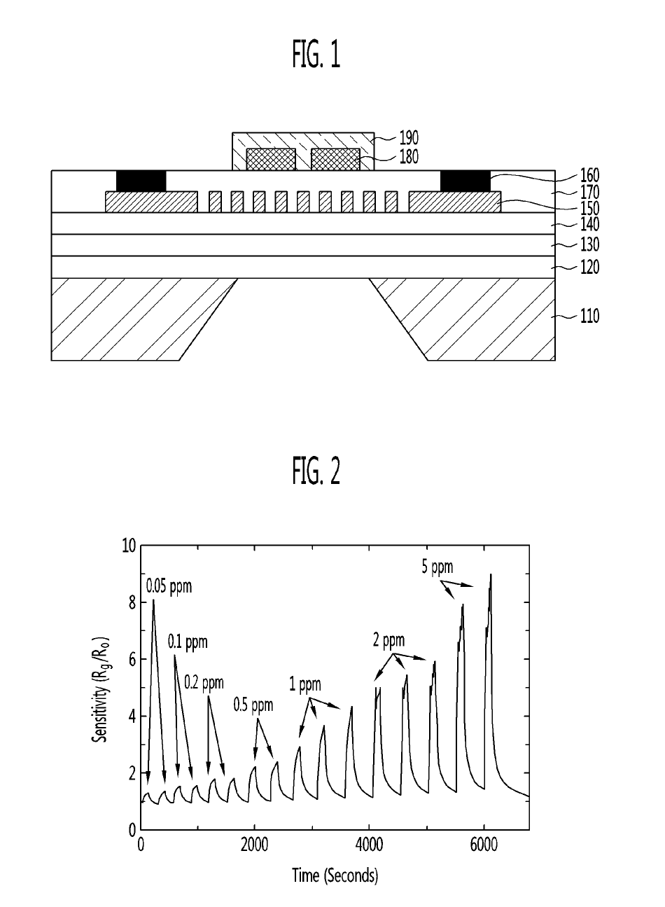

FIG. 1 is a cross-sectional view of a semiconductor gas sensor according to an exemplary embodiment of the present invention.

Referring to FIG. 1, the semiconductor gas sensor according to an exemplary embodiment of the present invention includes a first silicon oxide thin layer 120, a silicon nitride thin layer 130, a second silicon oxide thin layer 140, a heater 150, a heater electrode pad 160, an insulating layer 170, a sensing electrode 180, and a low-dimensional semiconductor nanomaterial 190, all of which are formed on a substrate 110.

The substrate 110 may employ a silicon substrate used in the typical semiconductor process, or a substrate into which aluminum oxide (Al₂O₃), magnesium oxide (MgO), quartz, gallium nitride (GaN), or gallium arsenide (GaAs) is doped. Meanwhile, a central rear surface of the substrate 110 on which the heater 150 is formed is etched and removed. To etch the rear surface of the substrate 110, a dry etching process using a photoresist pattern may be used.

The first silicon oxide thin layer 120, the silicon nitride thin layer 130, and the second silicon oxide thin layer 140 are sequentially stacked on the substrate 110, thereby forming a membrane.

The membrane serves as an anti-etching layer when the rear surface of the substrate 110 is etched, as well as a support of the heater 150.

Further, the membrane serves to prevent a device from being deformed by heat generated when the heater 150 is driven, and may be formed of a silicon oxide thin layer or a silicon nitride thin layer, or in a stacked structure of the silicon oxide thin layer and the silicon nitride thin layer. In the figure, an example where the membrane is formed in the stacked structure of the first silicon oxide thin layer 120, the silicon nitride thin layer 130, and the second silicon oxide thin layer 140 is shown. In this manner, the membrane may be formed of the silicon oxide thin layer having compressive stress and the silicon nitride thin layer having tensile stress in the structure as shown in FIG. 1. Alternatively, the membrane may be formed of only one of the thin layers.

The membrane may be formed using thermal oxidation, sputtering, or chemical vapor deposition.

The heater 150 serves to increase a surrounding temperature in order to improve a gas sensing characteristic, and may be formed of a material such as gold (Au), tungsten (W), platinum (Pt), or palladium (Pd). The heater 150 is formed on the membrane on the central region of the substrate 110, and may be formed in an inter-digital form or in a gap form.

The heater 150 may be formed using sputtering, electron beam, or evaporation.

Meanwhile, to enhance adhesion strength when the heater 150 is formed, an adhesive layer (not shown) may be additionally formed on the membrane using chromium (Cr) or titanium (Ti). The adhesive layer may be formed using sputtering, electron beam, or evaporation.

The heater 150 may be connected with an external circuit (not shown) by the heater electrode pad 160 and a bonding wire. The external circuit is set so as to operate the heater 150 only when the gas adsorbed to the sensing material is desorbed. Thereby, it is possible to reduce power consumption and obtain a rapid response characteristic. Alternatively, the heater 150 may be operated continuously regardless of the desorption of the gas.

The heater electrode pad 160 serves to transmit power to the heater 150, and may be contacted with a bonding wire (not shown) for connection with a power supply. Preferably, two heater electrode pads 160 are formed on opposite sides of the device. For example, the insulating layer 170 is etched to partially expose the surface of the heater 150, and then a conductive layer is buried in the etched region. Thereby, the heater electrode pad 160 may be formed.

Meanwhile, the heater electrode pad 160 may be formed of the same material as the heater 150, using sputtering, electron beam, or evaporation.

The insulating layer 170 is formed on the membrane so as to cover the heater electrode pad 160. Here, the insulating layer 170 is formed so that an upper portion of the heater electrode pad 160 is exposed to contact, for instance, a bonding wire. The insulating layer 170 is formed of a silicon oxide thin layer or a silicon nitride thin layer, and may be formed using thermal oxidation, sputtering, or CVD.

The sensing electrode 180 outputs a change in value of resistance, which is caused by the gas adsorbed to or desorbed from the sensing material, to the outside. The sensing electrode 180 is formed on the insulating layer 170 on the central region of the substrate 110. Preferably, a pair of sensing electrodes 180 are formed so as to cover the central region of the substrate. The sensing electrode 180 may be formed of platinum (Pt), aluminum (Al), or gold (Au) using sputtering, electron beam, or evaporation. Opposite ends of the sensing electrode 180 are in contact with a bonding wire (not shown) for transferring a signal.

The low-dimensional semiconductor nanomaterial 190 is a gas sensing material for adsorbing or desorbing the gas, and is formed on the sensing electrode 180 or the insulating layer 170 so as to cover the sensing electrode 180. The low-dimensional semiconductor nanomaterial 190 may employ nanopowder, nanowires, nanorods, CNTs, or graphene, and may be formed using a sol-gel method, drop coating, screen printing, or CVD.

The semiconductor gas sensor having the aforementioned configuration according to an exemplary embodiment of the present invention uses the low-dimensional semiconductor nanomaterial having a high-sensitivity characteristic at low temperature, particularly at room temperature, even with a small volume, and operates the heater only when the adsorbed gas is desorbed, thereby having a low power characteristic and a rapid response characteristic.

FIG. 2 is a graph showing measuring sensitivity of a semiconductor gas sensor according to an exemplary embodiment of the present invention.

As an experiment, the sensor was exposed to nitrogen dioxide (NO₂) gas of 0.05 ppm to 5 ppm every 3 minutes while power of 17 mW was continuously applied to the heater, and a change in resistance of the sensor caused by concentration of the NO₂ gas was measured. It took abut 3 minutes to desorb the adsorbed gas. Here, ZnO nanorods were used as a sensing material.

Generally, the ZnO material is an n-type semiconductor due to existence of oxygen vacancies, and the NO₂ gas is oxidative gas. Thus, as the concentration of gas increases, a quantity of conductive electrons increases, and so a change in value of the resistance of the sensor increases as well. This can be seen from FIG. 2.

Meanwhile, the heater of the present invention is a micro device that can be housed in a sensor fabricated by a MEMS process compatible with a CMOS process, can reach a desired temperature within a short time, and has durability in that its characteristics do not vary despite long-term use under various atmospheres.

When exposed to the air environment, the gas sensor reacts with nitrogen or oxygen, which occupies a larger fraction of the air than anything else. The nitrogen causes no reaction with the sensing material within the gas sensor because it is an inert gas, whereas the oxygen is adsorbed onto a surface of the sensing material and exists in the form of ions of O²⁻, O₂⁻, and O⁻. At this time, the oxygen receives electrons from the sensing material. In this way, an electron depletion layer deprived of the electrons has a thickness of tens of nm. In the case of a nanomaterial having the size of a conductive path similar to the electron depletion layer, when reacting with oxidative gas or reductive gas, it undergoes a very great change in electrically conductible region in the entire size of the conductive path. Thus, when the nanomaterial is exposed to the oxidative gas or the reductive gas, a very great change in resistance, i.e. a high-sensitivity characteristic, is shown. This characteristic is shown even at a low working temperature.

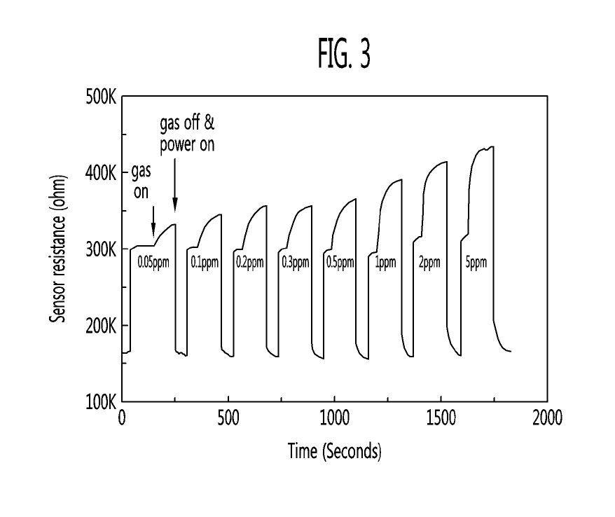

FIG. 3 is a graph showing measuring sensitivity of a semiconductor gas sensor according to another exemplary embodiment of the present invention.

As an experiment, the sensor was exposed to NO₂ gas of 0.05 ppm to 5 ppm every 2 minutes, and a change in resistance of the sensor caused by concentration of the NO₂ gas was measured. ZnO norods were used as a sensing material.

Unlike FIG. 2, the experiment was conducted without applying power to the heater when the sensor was exposed to the gas, and the power of 15 mW was applied to the heater only when the NO₂ gas adsorbed to the sensing material was desorbed.

Further, the sensor was placed in a chamber, and only nitrogen or air was injected. In the meantime, the NO₂ gas was gradually injected, and the change of resistance was measured

Referring to FIG. 3, it can be found that a resistance value of the sensor was constant when the NO₂ gas was not injected at room temperature, but increased after the sensor was exposed to the NO₂ gas.

Further, it can be found that the adsorbed NO₂ gas was desorbed when power was applied to the heater mounted in the sensor after the injection of the NO₂ gas was stopped, and thus the resistance was reduced. A process of desorbing the NO₂ gas was performed every 2 minutes. It can be found that, when the desorption of the NO₂ gas was completed, the sensor had initial resistance before the reaction with the NO₂ gas.

Comparing response times which it takes the sensor to sense the gas in FIGS. 2 and 3, it can be found that a time in which the resistance value converted by the gas sensing reaches saturation is much shorter in the operation mode of FIG. 3, and an operation time required to desorb the adsorbed gas is much shorter as well. Even when it is assumed that the operation time required to sense the gas and the operation time required to desorb the adsorbed gas are equal to each other, the overall power consumption of the sensor is reduced by about a half.

According to the present invention as described above, it is possible to improve a gas sensing characteristic using a low-dimensional semiconductor nanomaterial having a high-sensitivity characteristic at room temperature.

Further, since the semiconductor gas sensor exhibits a high-sensitivity characteristic at room temperature, it is possible to reduce power required to raise a surrounding temperature. Since the semiconductor gas sensor can desorb the gas adsorbed by a reaction at room temperature with still less energy within a short time, it is possible to remarkably reduce power consumption, and reduce an operation time at high temperature, which can increase durability of the sensor.

Also, it can drastically reduce a volume of the sensing material due to the high-sensitivity characteristic of the low-dimensional semiconductor nanomaterial. Thereby, it is possible significantly reduce power required to desorb the adsorbed gas and provide a rapid response characteristic.

In addition, no additional circuit is required due to a low power characteristic of the semiconductor gas sensor, and it is possible to guarantee long-term use even within limited cell capacity. It is possible to drive the semiconductor gas sensor using a self chargeable power supply of energy conversion devices such as solar elements, thermoelectric elements, and piezoelectric elements.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A semiconductor gas sensor having low power consumption, which adsorbs gas to a low-dimensional semiconductor nanomaterial at room temperature, outputs a change in resistance of the low-dimensional semiconductor nanomaterial, applies power to a heater, desorbs the gas adsorbed to the low-dimensional semiconductor nanomaterial, and returns the resistance of the low-dimensional semiconductor nanomaterial back to initial resistance.

2. The semiconductor gas sensor according to claim 1, comprising:
a membrane;
a substrate located at a lower portion of the membrane and having a central region etched such that a space between the substrate and the lower portion of the membrane is exposed;
a heater formed on the membrane at the central region of the substrate and driven when the gas adsorbed to the low-dimensional semiconductor nanomaterial is desorbed;
an insulating layer formed on the membrane so as to cover the heater;
a sensing electrode formed on the insulating layer at the central region of the substrate; and
the low-dimensional semiconductor nanomaterial formed on the sensing electrode.

3. The semiconductor gas sensor according to claim 1, wherein the low-dimensional semiconductor nanomaterial includes nanopowder, nanowires, nanorods, carbon nanotubes (CNTs), or graphene.

4. The semiconductor gas sensor according to claim 1, wherein the low-dimensional semiconductor nanomaterial is formed by a sol-gel method, a drop coating method, a screen printing method, or a chemical vapor deposition method.

5. The semiconductor gas sensor according to claim 1, wherein the membrane prevents a device from being deformed by heat generated when the heater is driven.

6. The semiconductor gas sensor according to claim 1, wherein the membrane is formed of a silicon oxide thin layer or a silicon nitride thin layer, or in a stacked structure of the silicon oxide thin layer and the silicon nitride thin layer.

7. The semiconductor gas sensor according to claim 1, wherein the membrane is formed in a single layered structure of a silicon oxide thin layer or a silicon nitride thin layer or in a multi-layered structure of the silicon oxide thin layer and the silicon nitride thin layer.
